(19) United States
(12) Patent Application Publication
(10) Pub. No.: US 2022/0039814 A1
(43) Pub. Date: Feb. 10, 2022

(54) MEDICAL ASPIRATION DEVICES AND METHODS

(71) Applicant: Shawn P. Fojtik, Park City, UT (US)

(72) Inventor: Shawn P. Fojtik, Park City, UT (US)

(21) Appl. No.: 17/395,438

(22) Filed: Aug. 5, 2021

Related U.S. Application Data
(60) Provisional application No. 63/061,785, filed on Aug. 5, 2020.

(51) Int. Cl. A61B 17/22; A61M 25/00

(52) U.S. Cl. CPC A61B 17/22; A61M 25/0068; A61B 2017/22038; A61B 2017/22079; A61M 25/0023

(57) ABSTRACT

A medical aspiration device includes an internal elongated element and a bulbous tip at a distal end of the internal elongated element. Together, the internal elongated element and the bulbous tip may define a disruptor of the medical aspiration device. The medical aspiration device may also include an outer elongated element, or a catheter, that includes a lumen within which the internal elongated element may be disposed and relative to which the bulbous tip may be extended and retracted. Extension and retraction of the bulbous tip may provide control over an aspiration force, or suction, that has been applied through the catheter to a distal tip of the catheter. Extension and retraction of the bulbous tip may disrupt matter to be aspirated by the medical aspiration device in a manner that facilitates aspiration of the matter.

### CROSS-REFERENCE TO RELATED APPLICATION

A claim for priority to the Aug. 5, 2020 filing date of U.S. Provisional Patent Application No. 63/061,785, titled MECHANICAL DILATOR, MACERATOR, ASPIRATOR AND METHODS TO START, STOP, STORE, AND DIRECT ASPIRATION FORCE (“the '785 Provisional Application”), is hereby made pursuant to 35 U.S.C. § 119(e). The entire disclosure of the '785 Provisional Application is hereby incorporated herein.

### TECHNICAL FIELD

This disclosure relates generally to the removal of matter, such as emboli, thrombi, plaques, pathology, and the like, from the body of a subject. More specifically, this disclosure relates catheters that may be used to aspirate and optionally disrupt matter within the body of a subject. Even more specifically, medical aspiration devices that mechanically disrupt matter as the matter is aspirated into a catheter and corresponding methods are disclosed.

### SUMMARY

A medical aspiration device is disclosed. A medical aspiration device according to this disclosure includes an internal elongated element and a bulbous tip at a distal end of the internal elongated element. Together, the internal elongated element and the bulbous tip may define a disruptor of the medical aspiration device. The medical aspiration device may be used with an outer elongated element, or a catheter, that includes a lumen within which the internal elongated element may be disposed and relative to which the bulbous tip may be extended and retracted.

The internal elongated element may comprise a tubular element, such as a hypotube, with a lumen extending therethrough. The lumen may have an inner diameter sufficient to receive a guide wire and an outer diameter small enough to enable fluids to flow between the internal elongated element and the catheter within which the internal elongated element is positioned.

The bulbous tip may be located at a distal end of the internal elongated element. An outer diameter of the bulbous tip may vary along the length of the bulbous tip. For example, a proximal end of the bulbous tip may have an outer diameter that enables it to fit within an opening to the lumen of the catheter. The bulbous tip or, more specifically, an outer surface and an outer diameter of the bulbous tip may taper outwardly relative to a longitudinal axis of the bulbous tip, from the proximal end or a proximal portion of the bulbous tip toward an intermediate portion or a distal portion of the bulbous tip. In some embodiments, the taper may be off-center. In embodiments where the taper is off-center, it may extend around only a portion of the circumference of the proximal portion the bulbous tip. Thus, a base of the circumference of the bulbous tip may be less tapered or untapered (i.e., the bulbous tip may lie flat along its base), while an upper portion of the circumference may include a more pronounced taper. In other embodiments, the proximal portion of the bulbous tip may include an isometrically centered, or circumferential, taper.

A maximum outer diameter of the bulbous tip, which may be located along the intermediate portion or the distal portion of the bulbous tip, may be at least as large as an inner diameter of a lumen of the catheter within which the internal elongated element is positioned. The outer diameter of the proximal end of the bulbous tip, the shape of the proximal portion of the bulbous tip, and the outer diameter of the intermediate potion or the distal portion of the bulbous tip may enable the bulbous tip to seal against a distal end of the catheter.

An internal passage of the bulbous tip may communicate with the lumen of the internal elongated element. In some embodiments, the internal passage of the bulbous tip may be coaxial with the lumen of the internal elongated element. In such embodiments, an inner diameter of the internal passage of the bulbous tip may enable the internal passage to receive a guide wire and, thus, enable movement of the bulbous tip along a length of the guide wire.

The distal portion of the bulbous tip may include a slant. The slant may be flat or concave. When the bulbous tip is extended from a distal tip of the catheter, an edge of the slant may be spaced apart from the distal tip of the catheter, providing an opening between the bulbous tip and the distal tip of the catheter or, at least, an opening between the slant of the bulbous tip and the distal tip of the catheter that is larger than openings between other portions of the bulbous tip and the distal tip of the catheter.

In some embodiments, the slant may include an opening, such as an aperture or a slot, that communicates with the internal passage within the bulbous tip and, thus, with a lumen of the internal elongated element. A position of the opening on the slant of the bulbous tip may enable it to be completely covered by the catheter when the internal elongated element is pulled into a completely retracted position relative to the catheter and exposed when the internal elongated element extends the bulbous tip distally from the distal tip of the catheter.

The intermediate portion of the bulbous tip may provide a transition between the proximal portion and a distal portion of the bulbous tip. The intermediate portion may have a substantially constant cross-sectional diameter along its length and, thus, a substantially constant outer diameter along its length. Alternatively, the outer diameter of the intermediate portion may gradually increase from its proximal side to a more central location, then decrease from the more central location to a distal side of the intermediate portion.

In embodiments where the bulbous tip includes both an intermediate portion and a distal portion, the distal portion may have any of a variety of suitable shapes. Without limitation, the distal portion of the bulbous tip may be blunt (i.e., flat), rounded, or tapered. The distal portion of a tapered bulbous tip or, more specifically, an outer surface and, thus, an outer diameter of the distal portion may taper downwardly relative to longitudinal axis of the bulbous tip, from a proximal side or proximal portion of the distal portion of the bulbous tip toward a distal end of the bulbous tip.

In another aspect, medical aspiration systems are disclosed. Such a system may include a medical aspiration device (i.e., an internal elongated element, a bulbous tip, and a catheter), an optional guidewire, an aspiration device, and, in some embodiments, a positioner.

In some embodiments, one or both of the internal elongated element and the catheter may include at least one bend, or curve. The bend of the catheter may impart the catheter with a planar aspect; the bend of the internal elongated element may likewise impart the internal elongated element with a planar aspect. When the internal elongated element is positioned within the lumen of the catheter, the internal elongated element may influence the bend of the catheter. The bend(s) of the catheter and/or the internal elongated element may be straightened out as the catheter and/or the internal elongated element resides on a guide wire. In embodiments where both the catheter and the internal elongated element include bends, the angle and/or shape of the bend of the internal elongated element, the longitudinal position of the bend of the internal elongated element relative to the longitudinal position of the bend of the catheter, and/or the rotational position of the bend of the internal elongated element relative to the rotational position of the bend of the catheter may influence, or alter, the bend of the catheter. Alteration of the bend(s) of the catheter and/or the internal elongated element may enable steering of the catheter and/or the internal elongated element.

The positioner of the medical aspiration system may be associated with a proximal portion of the internal elongated element and a proximal portion of the catheter in a manner that enables the internal elongated element to move distally and proximally through the catheter. Thus, the positioner may enable the bulbous tip to extend at least partially from and be retracted partially into the distal tip of the catheter. The positioner may be spring loaded in such a way that once the internal elongated element and the distal tip are forced distally relative to the catheter, a spring automatically returns, or forces, the internal elongated element and the bulbous tip to their initial positions relative to the catheter.

According to another aspect of this disclosure, an aspiration method may include positioning a guide wire within a subject's body and introducing a medical aspiration device into the subject's body along the guide wire. The medical aspiration device may be introduced into the subject's body as an assembly including a disruptor and a catheter over the disruptor. Alternatively, the catheter may be introduced into the subject's body over an internal elongated element of the disruptor. With the catheter and the disruptor in place, an aspiration force, or suction, may be applied to the catheter. As the aspiration force is applied with a bulbous tip of the disruptor extended distally from a distal tip of the catheter, fluids and/or other matter (e.g., emboli, thrombi, plaques (e.g., atheroma, etc.), pathology (e.g., tumors, polyps, cysts, etc.), organs, tissue, etc.) may be drawn between a proximal portion of the bulbous tip and an opening at the distal tip of the catheter. The bulbous tip may be moved relative to the distal tip of the catheter in a manner that disrupts the fluid and/or other matter, facilitating aspiration of the fluid and/or other matter into the catheter. For example, repeated extension and retraction, or back-and-forth movement, of the bulbous tip may disrupt (e.g., soften, macerate, etc.) matter as it is aspirated into the opening at the distal catheter.

Other aspects of the disclosed subject matter, as well as features and advantages of various aspects of the disclosed subject matter, should become apparent to those of ordinary skill in the art through consideration of the ensuing description, the accompanying drawings, and the appended claims.

### BRIEF DESCRIPTION OF THE DRAWINGS

In the drawings:

FIG. 1 is perspective view of an embodiment of a disruptor of a medical aspiration device according to this disclosure, with the disruptor including an internal elongated element and a bulbous tip at a distal end of the internal elongated element;

FIG. 2 is a side view of the embodiment of disruptor depicted by FIG. 1, with an embodiment of a positioner 60 on a proximal end of the internal elongated element of the disruptor;

FIG. 3 is a perspective view of an embodiment of a medical aspiration device or system that includes the embodiment of disruptor shown in FIGS. 1 and 2, the embodiment of positioner depicted by FIG. 2, and a catheter;

FIG. 4 is a partial perspective view showing a distal end of the embodiment of medical aspiration device or system illustrated by FIG. 3, with the bulbous tip of the disrupter in a retracted arrangement relative to a distal tip of the catheter;

FIG. 5 is a partial perspective view showing the distal end of the embodiment of medical aspiration device or system illustrated by FIG. 3, with the bulbous tip of the disrupter in a partially extended arrangement, or a partially open arrangement, relative to the distal tip of the catheter;

FIG. 6 is a partial perspective view showing the distal end of the embodiment of medical aspiration device or system illustrated by FIG. 3, with the bulbous tip of the disrupter in a fully extended arrangement, or a completely open arrangement, relative to the distal tip of the catheter;

FIGS. 7A and 7B show another embodiment of a bulbous tip of a disruptor or a medical aspiration device or system, in which a proximal portion of the bulbous tip is tapered differently that the proximal portion of the embodiment of bulbous tip shown in FIGS. 1-6;

FIGS. 8 and 9 are perspective view of disruptors with embodiments of bulbous tips with distal portions that differ from the distal portion of the embodiment of bulbous tip shown in FIGS. 1-6;

FIG. 10 depict an embodiment of medical aspiration device in which the internal elongated element and the catheter include bends, showing the bends in longitudinal and rotational alignment with each other;

FIG. 11 depicts the effect of a guide wire on the embodiment of medical aspiration device shown in FIG. 10;

FIG. 12 depicts an example of the effect of misalignment of the internal elongated element and the catheter on the bend of the catheter;

FIGS. 13A and 13B are partial perspective views of the distal end of an embodiment of medical aspiration device respectively showing a bulbous tip of the medical aspiration device in fully extended and partially extended arrangements relative to a catheter of the medical aspiration device; and

FIG. 14 schematically depicts the delivery of a substance to a site at which a bulbous tip of a medical aspiration device is located.

### DETAILED DESCRIPTION

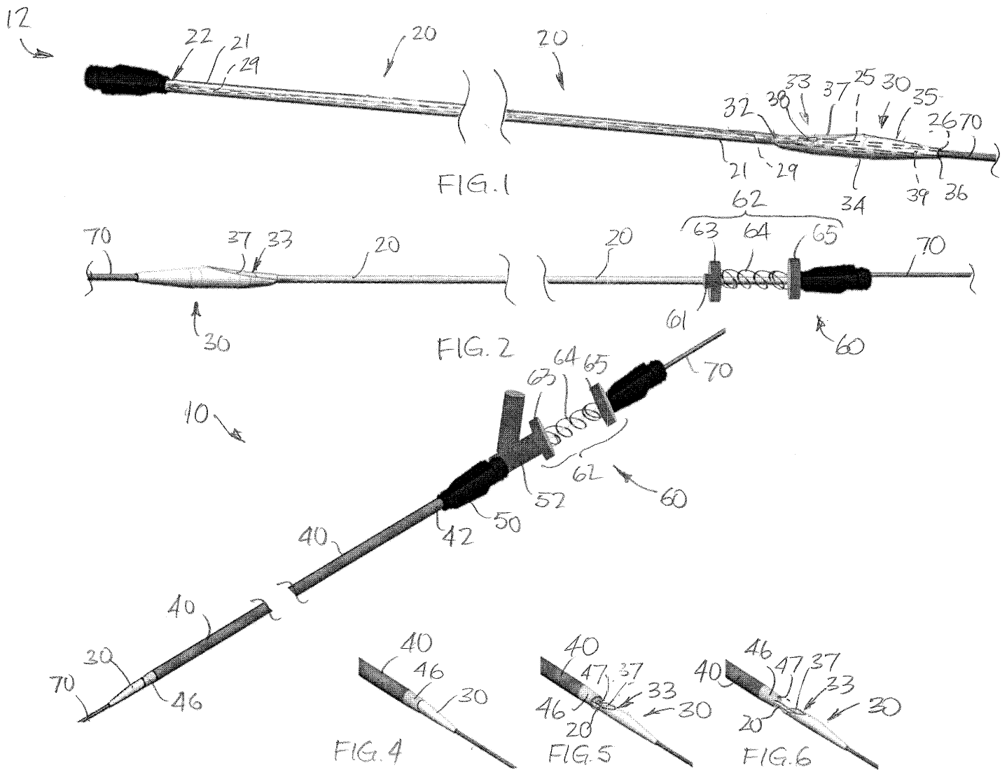

FIGS. 1-3 show an embodiment of a medical aspiration device 10 according to this disclosure. As shown in FIG. 1, the medical aspiration device 10 includes a disruptor 12 that comprises an internal elongated element 20 and a bulbous tip 30. The disruptor 12 may also include a connector 50 that facilitates placement of the disruptor 12 in fluid communication with other components. In addition, as shown in FIG. 3, the medical aspiration device 10 includes an outer element, such as a catheter 40, with which the disruptor 12 may be assembled. In some embodiments, the medical aspiration device may also include a positioner 60, as illustrated by FIGS. 2 and 3.

The internal elongated element 20 may have a tubular configuration and, thus, include a wall 21 and a lumen 29 defined by the wall 21 and extending through the length of the internal elongated element 20. An inner diameter (ID) of the internal elongated element 20, or the diameter of its lumen 29, may enable the internal elongated element to receive and move over a length of a guide wire 70. An outer diameter (OD) of the internal elongated element 20 may enable it to be received by and to move longitudinally through a lumen of the catheter 40 and to enable fluids and/or other matter (e.g., emboli, thrombi, plaques (e.g., atheroma, etc.), pathology (e.g., tumors, polyps, cysts, etc.), organs, tissue, etc.) to flow between the internal elongated element 20 and the catheter or tube within which the internal elongated element is positioned. Without limitation, the inner diameter of the internal elongated element 20 may be as small as about 0.010 inch (about 0.254 mm) (e.g., for use with a 0.010 inch (0.254 mm) guide wire 70, etc.) and the outer diameter of the internal elongated element 20 may be as small as about 0.015 inch (about 0.381 mm). The inner diameter of the internal elongated element 20 may be as large as about 0.040 inch (about 1.02 mm) (e.g., for use with a 0.038 inch (0.965 mm) guide wire 70, etc.) and the outer diameter of the inner elongated element 20 may be as large as about 0.045 inch (about 1.14 mm). In some embodiments, the internal elongated element 20 may have even larger dimensions.

The internal elongated element 20 may comprise a single piece or a plurality of pieces. A metal, a polymer, or a combination of a metal and a polymer may be used to form the internal elongated element. In a specific embodiment, the internal elongated element 20 may comprise a hypotube, which may be formed from a stainless steel or a nitinol. Such a hypotube may include features (e.g., spiral or circumferential slits, or cuts, such as those disclosed by U.S. Patent Application Publication US 2019/0232018 A1 (“the '018 Publication”), the entire disclosure of which is hereby incorporated herein, etc.) that enhance its flexibility. In such embodiments, the internal elongated element may also include one or more polymer coatings, which may be carried externally over and/or internally within a wall of the hypotube, as also disclosed by the '018 Publication).

At least a portion of the outer surface of the internal elongated element 20 may facilitate aspiration of matter into and through a lumen 49 of a catheter 40 within which the internal elongated element 20 resides. Without limitation, the outer surface of the internal elongated element 20 may include disruptive features. The disruptive features may disrupt aspirated matter in a manner that facilitates the continued, uninterrupted movement of such aspirated matter proximally through the lumen 49 of the catheter 40. Examples of disruptive features include, but are not limited to, blades, fins or wings, other raised features (e.g., grating edges, blades, etc.), fenestrations, and/or the like.

The internal elongated element 20 has a proximal end 22 and a distal end 26.

The bulbous tip 30 of the medical aspiration device 10 may be located at a distal end 26 of the internal elongated element 20. The embodiment of bulbous tip 30 depicted by FIGS. 1-3 includes, from its proximal side to its distal side, a proximal end 32, a proximal portion 33, an intermediate portion 34, a distal portion 35, and a distal end 36.

In some embodiments, an entirety of the bulbous tip 30 may be positioned over a distal portion 25 of the internal elongated element 20 (i.e., the distal portion 25 of the internal elongated element 20 may extend through the bulbous tip 30). In such embodiments, the distal end 26 of the internal elongated element 20 may be coextensive with the distal end 36 of the bulbous tip 30. In other embodiments, the bulbous tip 30 may be secured to the distal end 26 of the internal elongated element 20 (i.e., the distal portion 25 of the internal elongated element 20 may not extend into or may extend only partially through the bulbous tip 30).

In embodiments where the distal portion 25 of the internal elongated element 20 extends into the bulbous tip 30, the distal portion 25 may be flared to provide for mechanical coupling between the distal portion 25 and the bulbous tip 30. As an alternative or in addition, the distal portion 25 may include other features, such as slots, flanges or other protrusions, and/or the like, to facilitate mechanical coupling of the bulbous tip 30 to the distal portion 25.

The bulbous tip 30 may include an internal passage 39 that communicates with a lumen 29 of the internal elongated element 20 or comprises a distal portion of the lumen 29 of the internal elongated element 20. The internal passage 39 and the lumen 29 may be coaxial. An inner diameter of the internal passage 39 may enable the internal passage 39 to receive and track over a guide wire 70 and, thus, enable movement of the bulbous tip 30 along a length of the guide wire 70.

A shape and dimensions of the bulbous tip 30 may enable it to be at least partially retracted into an opening 47 in a distal tip 46 of the catheter 40, as shown in FIG. 4. As the bulbous tip 30 or, more specifically, a proximal portion 33 of the bulbous tip 30 is retracted into the opening 47 in the distal tip 46 of the catheter 40, the proximal portion 33 of the bulbous tip 30 may seal against the distal tip 46 of the catheter 40.

With continued reference to FIGS. 1-3, at and near the proximal end 32 of the bulbous tip 30, an outer diameter of the proximal portion 33 may be smaller than an inner diameter of the opening 47 and the lumen 49 of catheter 40, enabling at least the proximal side of the proximal portion 30 to be pulled into the opening 47 and the lumen 49. In some embodiments, the entire proximal portion 33 of the bulbous tip 30 may be retracted into the opening 47 and lumen 49 of the catheter 40. In other embodiments, distal locations along the length of the proximal portion 33 may have outer diameters that equal or exceed the inner diameter of the opening 47 and the lumen 49, enabling the proximal portion 33 of the bulbous tip 30 to abut and optionally seal against the distal tip 46 of the catheter 40.

The proximal portion 33 of the bulbous tip 30 may taper outwardly relative to a longitudinal axis of the bulbous tip 30, from the proximal end 32 to the intermediate portion 34. The taper may provide a smooth transition between the outer surface of the internal elongated element 20 and the proximal portion 33 of the bulbous tip 30, which may enable the internal elongated element 20 to be pulled proximally and the proximal portion 33 to be retracted into the opening 47 in the distal tip 46 of the catheter 40 or the opening 47 in the distal tip 46 of the catheter 40 to be smoothly pushed over the proximal portion 33 of the bulbous tip 30. The taper may be a low angle taper (e.g., about 5° to about 40°, etc.).

The taper of the proximal portion 33 may be off-center. In embodiments where the taper is off-center, it may extend around only a portion of the circumference of the proximal portion 33 the bulbous tip 30. Thus, the proximal portion 33 of the bulbous tip 30 may include a slightly tapered or untapered base and a more tapered top, enabling the proximal portion 33 to lie substantially flat (accounting for a slight taper) or completely flat (accounting for no taper) along its base. Such a taper may enable the bulbous tip 30 to focus aspiration forces, or suction, applied through the lumen 49 of the catheter 40 to one side of the opening 47 in the distal tip 46 of the catheter 40.

Alternatively, as shown in FIGS. 7A and 7B, the taper of a proximal portion 33′ of a bulbous tip 30′ may comprise an isometrically centered, or circumferential, taper, imparting the proximal portion 33′ with a somewhat conical or frustoconical shape. A circumferential taper may enable the bulbous tip 30′ to focus aspiration forces applied through the lumen 49 of the catheter 40 in an annular fashion around the outer edges of the opening 47 in the distal tip 46 of the catheter 40.

With reference again to FIGS. 1-3, the proximal portion 33 may include a relatively flat section, which may be referred to as a “slant 37,” along a portion of the length of the proximal portion 33. The slant 37 may be flat or somewhat concave. A location of the slant 37 on the proximal portion 33 may facilitate its complete introduction into the opening 47 and the lumen 49 of the catheter 40 when the disruptor 12 and its bulbous tip 30 are fully retracted relative to the catheter 40. For example, locations along the proximal portion 33 that have the same or greater outer diameters than the opening 47 and the lumen 49 of the catheter 40 may be located distal to the distal-most extent of the slant 37.

In embodiments where the slant 37 is concave, the concave shape may facilitate direction of suction to a target location within the subject's body and/or shape the suction that is directed to the target location. The edges defined by a concave slant 37 may facilitate disruption (e.g., agitation, debulking, grasping, biting, shaving, cutting, maceration, etc.) of matter as the matter is aspirated between the slant 37 and the outer edges of the opening 47 through the distal tip 46 of the catheter 40 and the proximal portion 33 of the bulbous tip 30 is retracted (once, repeatedly, etc.) into the opening 47 in the distal tip 46.

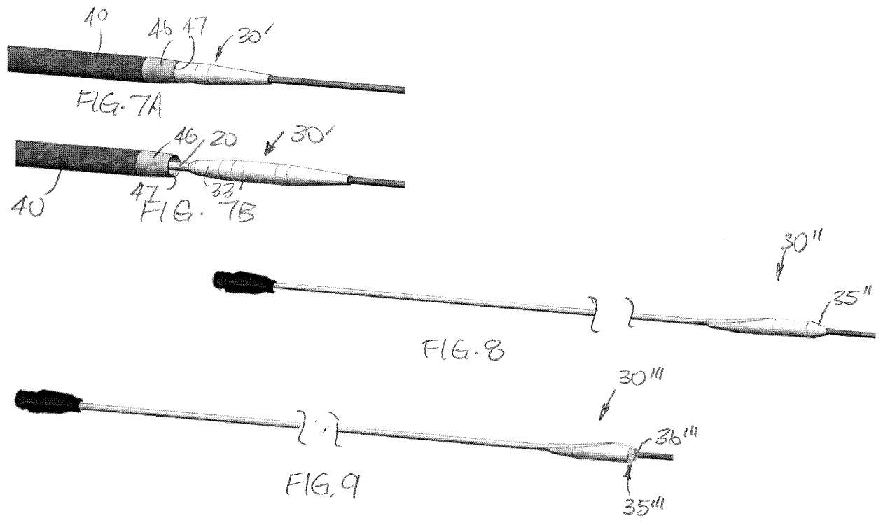

The slant 37 may include an opening 38 (e.g., an aperture, a slot, etc.) that communicates with the internal passage 39 within the bulbous tip 30 and, thus, with the lumen 29 of the internal elongated element 20. A position of the opening 38 on the slant 37 of the bulbous tip may enable the opening 38 to be received and optionally sealed within the catheter 40 when the bulbous tip 30 is pulled into a completely retracted position relative to the catheter 40. Conversely, the opening 38 may be exposed when the bulbous tip 30 extends distally relative to the distal tip 46 of the catheter 40, as shown in FIG. 5 (partial distal extension of the bulbous tip 30) and FIG. 6 (full distal extension of the bulbous tip 30).

In some embodiments, the slant 37 may include fenestrations, raised features (e.g., grating edges, blades, etc.), or the like that may disrupt aspirated matter in a manner that facilitates the continuous, uninterrupted proximal flow of such aspirated matter into the opening 47 in the distal tip 46 of the catheter 40 and through the lumen 49 of the catheter 40.

Continuing with reference to FIGS. 1-3, the intermediate portion 34 of the bulbous tip 30 may provide a transition between the proximal portion 33 and the distal portion 35 of the bulbous tip 30. As illustrated, the outer diameter of the intermediate portion 34 may gradually increase from its proximal side to a more central location, then decrease from the more central location to a distal side of the intermediate portion, with the central location having the largest outer diameter, or maximum outer diameter, of the bulbous tip 30. A location on the proximal side of the intermediate portion 34 where the outer diameter of the intermediate portion 34 equals or exceeds the inner diameter of the opening 47 and the lumen 49 of the catheter may abut and optionally seal against the distal tip 46 of the catheter 40. Alternatively, in embodiments where the proximal portion 33 of the bulbous tip 30 abuts and optionally seals against the distal tip 46 of the catheter 40, the intermediate portion 34 may have a substantially constant outer diameter, which may also comprise the maximum outer diameter of the bulbous tip 30. The maximum outer diameter of the bulbous tip 30 may be about the same as or less than the outer diameter of the distal tip 46 of the catheter. In some embodiments, including those where the outer diameter of the distal tip 46 of the catheter is tapered, the maximum outer diameter of the bulbous tip 30 may be less than the outer diameter along a majority of the length of the catheter 40, or than the outer diameter of the catheter 40.

The distal portion 35 of the bulbous tip 30 may be tapered inwardly relative to the longitudinal axis of the bulbous tip 30, from the intermediate portion 34 to the distal end 36 of the bulbous tip 30. The taper of the distal portion 35 may mirror the taper of the proximal portion 33 or it may differ from the taper of the proximal portion 33. The taper may be a low angle taper (e.g., about 5° to about 40°, etc.). Such a taper may enable the bulbous tip 30 to track over a guide wire 70 within the body of a subject.

The taper of the distal portion 35 of the bulbous tip 30 may be off-center. In embodiments where the taper is off-center, it may extend around only a portion of the circumference of the proximal portion 33 the bulbous tip 30. Thus, the proximal portion 33 of the bulbous tip 30 may include a slightly tapered or untapered base and a more tapered top, enabling the proximal portion 33 to lie substantially flat (accounting for a slight taper) or completely flat (accounting for no taper) along its base. Alternatively, as shown in FIG. 7, the taper of the distal portion 35 may comprise a circumferential taper, imparting the distal portion 35 with a somewhat conical or frustoconical shape.

As an alternative to the just-described gradually tapered distal portion 35, the distal portion 35″ of the bulbous tip 30″ may have a steep (e.g., ≥45°) taper, as shown in FIG. 8, or the distal portion 35′″ of the bulbous tip 30′″ may define a rounded or blunt distal end 36″, as shown in FIG. 9. Such embodiments may be useful in certain situations, such as where anatomy limits the extent to which the distal end 36 may be advanced beyond matter to be aspirated (i.e., in situations with a short distal purchase).

Referring still to FIGS. 1-3, a material or materials of the bulbous tip 30 may enable it to seal against the distal tip 46 of the catheter 40 as the proximal portion 33 of the bulbous tip 30 is retracted into the opening 47 in the distal tip 46, which may enable the bulbous tip 30 to control the communication of an aspiration force, or suction, from within the lumen 49 of the catheter 40 to locations beyond the distal tip 46 of the catheter 40. Thus, the material(s) of the bulbous tip 30 may render it inflexible, or impart it with rigidity, and, optionally, impart a surface of the proximal portion 33 with some compliance. In some embodiments, the bulbous tip 30 may comprise a polymer. Suitable polymers include polyethylene (PE), polypropylene (PP), and fluorinated ethylene propylene (FEP), among others. Alternatively, the bulbous tip 30 may be made from a metal.

The bulbous tip 30 may manufactured separately from the internal elongated element 20 and then assembled with and secured to the distal end 26 of the internal elongated element 20. The bulbous tip 30 may be secured to the distal end 26 in any suitable manner (e.g., mechanically, adhesively, with welds, etc., or any combination of the foregoing).

Alternatively, in embodiments where the bulbous tip 30 is formed from a polymer, the bulbous tip 30 may be molded in place on the distal end 26 of the internal elongated element 20.

As another alternative, the internal elongated element 20 and bulbous tip 30 may be manufactured as an integral unit. For example, in embodiments where the internal elongated element 20 and bulbous tip 30 are formed from a polymer, they may be coextruded or molded. As another example, the internal elongated element and bulbous tip 30 may be formed by modifying (e.g., cutting, machining, expanding, compressing, etc.) different sections of the same hypotube.

The catheter 40 may have a tubular configuration and, thus, include a wall 41 and a lumen 49 defined by the wall 41. The lumen 49 may extend through the length of the catheter 40, from a proximal end 42 to the distal tip 46 thereof. The lumen 49 may communicate through the opening 47 in the distal tip 46.

An inner diameter (ID) of the catheter 40, or the diameter of its lumen 49, may enable the catheter 40 to receive the internal elongated element 20 and enable the internal elongated element 20 to move longitudinally through the lumen 49. The inner diameter of the catheter 40 may also enable fluids and other matter to be aspirated between outer surfaces of the internal elongated element 20 and the surfaces of the lumen 49, or the inner surfaces of the catheter 40. An outer diameter (OD) of the catheter 40 may enable it to be inserted into and moved through a desired opening (e.g., vessel, other hollow organ, tube, cavity, etc.) within the body of a subject. Without limitation, the inner diameter of the catheter 40 may be as small as about 0.015 inch (about 0.381 mm) and the outer diameter of the catheter 40 may be as small as about 0.020 inch (about 0.508 mm). The inner diameter of the catheter 40 may be as large as about 0.045 inch (about 1.14 mm) and the outer diameter of the catheter 40 may be as large as about 0.050 inch (about 1.27 mm). In some embodiments, the catheter 40 may have even larger dimensions.

In some embodiments, the catheter 40 is may be 8F (i.e., 0.105 inch; 2.67 mm) or larger (e.g., 10F, 11F, etc.) in size. As an example, a 10F or an 11F catheter with an inner diameter of 0.125 inch (3.175 mm) has a lumen 49 with a cross-sectional area of 0.0122 in² (7.88 mm²). If such a catheter 40 is placed over an inner elongated element 20 with an outer diameter of 0.035 inch (0.889 mm) (i.e., a cross-sectional area of 0.001 in² or 0.62 mm²), the remaining cross-sectional area through which aspirated matter may flow is still about 0.011 in² (7.26 mm²). Similarly, if such a catheter 40 is placed over an inner elongated element 20 with an outer diameter of 0.042 inch (1.07 mm) (i.e., a cross-sectional area of 0.0014 in² or 0.90 mm²), the remaining cross-sectional area through which aspirated matter may flow is still about 0.010 in² (6.98 mm²). Accordingly, the reduction in area and volume for aspirated fluid and/or other matter to flow is only reduced by about 5% when the larger internal elongated element 20 is used, enabling the use of a larger (e.g., 0.035 inch, etc.) guide wire 70, which may improve trackability while minimizing any reduction in flow.

The connector 50 of the medical aspiration device 10 may be coupled to the proximal end 42 of the catheter 40. The connector 50 may comprise any suitable type of catheter connector known in the art, and it may facilitate coupling of the catheter 40 to other devices or equipment, including without limitation the disruptor 12 and an aspiration device of a type known in the art (e.g., a syringe, an aspirator, wall suction, an electromechanical aspiration pump, etc.).

In some embodiments, a Y-adapter 52 may be coupled to the connector 50 to enable communication to be established between the aspiration device and the lumen 49 of the catheter 40 and, thus, to enable an aspiration force to be applied to the lumen 49 of the catheter 40.

The positioner 60 of the medical aspiration device 10 may be coupled to the proximal end 22 of the internal elongated element 20 of the disruptor 12. More specifically, the positioner 60 may include a coupler 61 that couples to the positioner 60 to the Y-connector 52 and/or the connector 50 on the proximal end 42 of the catheter 40. The connector 50 and the Y-connector 52 may seal against an outer surface of the internal elongated element 20 (e.g., with a hemostasis valve, etc.).

In addition, the positioner 60 includes an actuator 62 that enables selective longitudinal movement of one or both of the internal elongated element 20 and the catheter 40 relative to the other. Stated another way, the actuator 62 may cause the internal elongated element 20 to move distally and proximally through the lumen 49 of the catheter 40 and/or the actuator 62 may cause the catheter 40 to move proximally and distally over the internal elongated element 20. Thus, the actuator 62 may cause the bulbous tip 30 to extend distally apart from the distal tip 46 of the catheter 40 and the actuator 62 may cause the bulbous tip 30 to be at least partially retracted into the distal tip 46 of the catheter 40.

In the embodiment depicted by FIGS. 2 and 3, the actuator 62 includes distal side 63, a spring 64, and a proximal side 65.

The distal side 63 of the actuator 62 may be positioned adjacent to, secured to, or continuous with the coupler of the positioner 60. A position of the distal side 63 may be fixed relative to a length of the catheter 40, but the internal elongated element 20 may slide through the distal side 63. Thus, as the distal side 63 is pulled closer to the proximal side 65, the catheter 40 is pulled proximally over the internal elongated element 20.

A position of the proximal side 65 of the actuator 62 may be fixed relative to a length of the internal elongated element 20; the proximal side 65 may be coupled to the internal elongated element 20. Thus, as the proximal side 65 is forced toward the distal side 63, the internal elongated element 20 is also forced distally through the lumen 49 of the catheter 40. Thus, movement of the distal side 63 and the proximal side 65 toward each other causes the bulbous tip 30 to extend distally from the distal tip 46 of the catheter 40, as shown in FIG. 5 (partial distal extension of the bulbous tip 30) and FIG. 6 (full distal extension of the bulbous tip 30). Conversely, movement of the distal side 63 and the proximal side 65 apart from each other causes the proximal portion 33 of the bulbous tip 30 to move toward and to be retracted into the distal tip 46 of the catheter 40, as shown in FIG. 4.

The spring 64 may be positioned between the distal side 63 and the proximal side 65 to force, or bias, them apart from one another. Thus, the spring 64 may ensure that, upon releasing a force that is intended to cause the bulbous tip 30 to extend distally from the distal tip 46 of the catheter 40, the proximal portion 33 of the bulbous tip 30 will be retracted into and optionally seal against the distal tip 46 of the catheter 40 (e.g., with a hemostasis valve, etc.).

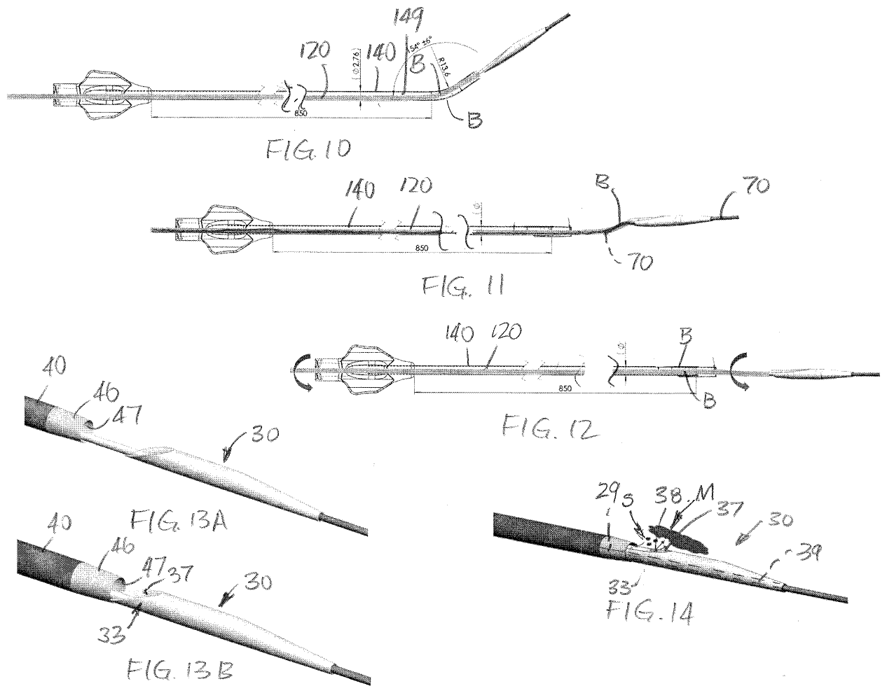

Turning now to FIGS. 10-12, in some embodiments, one or both of the internal elongated element 120 and the catheter 140 may include at least one bend B, or curve. FIG. 10 illustrates an embodiment in which a distal portion 145 of the catheter 140 is curved and, thus, includes a bend B. The internal elongated element 120 also has a bend B.

The bend B of the catheter 140 may impart the catheter 140 with a planar aspect; the bend B of the internal elongated element 120 may likewise impart the internal elongated element 120 with a planar aspect. When the internal elongated element 120 is positioned within the lumen 149 of the catheter 140, the internal elongated element 120 may influence the bend B of the catheter 140. The bend(s) B of the catheter 140 and/or the internal elongated element 120 may be straightened out as the catheter 140 and/or the internal elongated element resides on a guide wire 70, as shown in FIG. 11. In embodiments where both the catheter 140 and the internal elongated element 120 include bends B, the angle and/or shape of the bend B of the internal elongated element 120, the longitudinal position of the bend B of the internal elongated element 120 relative to the longitudinal position of the bend B of the catheter 140, and/or the rotational position of the bend B of the internal elongated element 120 relative to the rotational position of the bend B of the catheter 140 may influence, or alter, the bend B of the catheter 140.

In the arrangement depicted by FIG. 10, the bend B of the internal elongated element 120 is both longitudinally aligned and rotationally aligned (i.e., their planar extents are substantially coplanar) with the bend B of the catheter 140.

When one or both of the internal elongated element 120 and catheter 140 is rotated relative to the other, as shown in FIG. 12, their planar extents diverge, influencing, or modifying, their bends B. Specifically, FIG. 12, shows a 180° rotation of the internal elongated element 120 relative to the catheter 140, which may cause the bend B of the catheter 140 to straighten somewhat, as depicted, or even to rotate 180°, depending on the relative rigidities of the catheter 140 and the internal elongated element 120.

Alteration of the bend(s) B of the catheter 140 and/or the internal elongated element 120 may enable steering of the catheter 140 and/or the internal elongated element 120.

As shown in FIG. 3, the disruptor 12 and the catheter 40 may be pre-assembled with each other before their use. While in an assembled arrangement, the disruptor 12 and the catheter 40 may be assembled with a guide wire 70, introduced over the guide wire 70 into the body of a subject, and advanced through the body of the subject until the bulbous tip 30 reaches a desired location, or a target location, within the body of the subject.

An aspiration method may include positioning a guide wire 70 within a subject's body and introducing a medical aspiration device 10 into the subject's body along the guide wire 70. The medical aspiration device 10 may be introduced into the subject's body. The medical aspiration device 10 may be introduced with the disruptor 12 and the catheter 40 in an assembled relationship. Alternatively, disruptor 12 may be introduced, and then the catheter 40 may be introduced over the disruptor 12.

With the catheter 40 and the disruptor 12 in place, an aspiration force, or suction, may be applied to the catheter 40; for example, with an aspiration device that communicates through a Y-adapter 52 with the lumen 49 of the catheter 40. The positioner 60 may initially hold the bulbous tip 30 of the disruptor 12 in its retracted arrangement relative to the distal tip 46 of the catheter 40, preventing the aspiration force from being communicated through the opening 47 in the distal tip 46 of the catheter 40.

Once aspiration is desired, the positioner 60 may be actuated to extend the bulbous tip 30 from the distal tip 46 of the catheter 40, enabling the aspiration force to be communicated distally beyond the distal tip 46. As the aspiration force is applied, fluids and/or other matter (e.g., emboli, thrombi, plaques (e.g., atheroma, etc.), pathology (e.g., tumors, polyps, cysts, etc.), organs, tissue, etc.) may be drawn between the proximal portion 33 of the bulbous tip 30 and the opening 47 in the distal tip 46 of the catheter 40.

The extent to which the aspiration force is applied, as well as a direction in which the aspiration force is applied, may be controlled. As illustrated by FIG. 13A, when the bulbous tip 30 is fully extended from the distal tip 46 of the catheter, the aspiration force may be applied over the full area of the opening 47 through the distal tip 46. In contrast, when the bulbous tip 30 is only partially extended, as illustrated by FIG. 13B, the size of the opening through which the aspiration force may be applied is smaller, increasing the force. Additionally, the slant 37 on the proximal portion 33 of the bulbous tip 30 may control or influence the direction in which the aspiration force is applied.

In some embodiments, the bulbous tip 30 may be moved relative to the distal tip 46 of the catheter 40 in a manner that disrupts the fluid and/or other matter, facilitating aspiration of the fluid and/or other matter into the catheter 40. For example, repeated extension (FIGS. 5 and 6) and retraction (FIG. 4), or back-and-forth movement, of the bulbous tip 30 may disrupt (e.g., agitate, soften, macerate, etc.) the matter, facilitating its aspiration into the opening 47 in the distal tip 46.

In embodiments where an opening 38 is provided in the proximal portion 33 of the bulbous tip 30 (e.g., in the slant 37 of the proximal portion 33, etc.), substances S that may facilitate aspiration of fluid and/or other matter from a particular site may be infused through the lumen 29 of the internal elongated element 20, the passageway 39 in the bulbous tip 30, and the opening 38 into that site, as shown in FIG. 14. Such substances S may reduce a viscosity of, dissolve, or lubricate the matter M to be aspirated. Alternatively, the substances S that are infused through the opening 38 may deliver therapy to the site at which the bulbous tip 30 is located, facilitate diagnostics at the site, or perform other functions.

Although the preceding disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A medical aspiration device, comprising:
an internal elongated element including a lumen with an inner diameter sufficient to receive a guide wire and an outer diameter small enough to enable fluids to flow between the internal elongated element and a catheter within which the internal elongated element is positioned;
a bulbous tip at a distal end of the internal elongated element, the bulbous tip having a maximum outer diameter that is at least as large as an inner diameter of a lumen of the catheter within which the internal elongated element is positioned to enable a portion of the bulbous tip to seal against the catheter at the distal end.

2. The medical aspiration device of claim 1, wherein the internal elongated element comprises a hypotube.

3. The medical aspiration device of claim 2, wherein the hypotube comprises a metal hypotube.

4. The medical aspiration device of claim 3, wherein the metal hypotube comprises a shape memory alloy.

5. The medical aspiration device of claim 4, wherein the shape memory alloy includes at least one bend.

6. The medical aspiration device of claim 1, wherein the bulbous tip includes a proximal side that seals against a distal end of a catheter within which the internal elongated element is positioned.

7. The medical aspiration device of claim 1, further comprising:
a positioner associated with a proximal portion of the internal elongated element to enable proximal and distal movement of the internal elongated element and the bulbous tip.

8. The medical aspiration device of claim 7, wherein the positioner is spring loaded to force the elongate element and the bulbous tip proximally.

9. The medical aspiration device of claim 1, wherein a proximal side of the bulbous tip includes a slant.

10. The medical aspiration device of claim 1, wherein a proximal side of the bulbous tip includes a circumferential taper.

11. The medical aspiration device of claim 1, wherein a proximal side of the bulbous tip includes one or more disrupting features.

12. The medical aspiration device of claim 1, wherein the bulbous tip includes a lumen with an inner diameter sufficient to receive the guide wire.

13. A medical aspiration system, comprising:
a guide wire;
a catheter including a lumen with an inner diameter;
an internal elongated element including a lumen with an inner diameter sufficient to receive the guide wire and an outer diameter small enough to enable fluids to flow between the internal elongated element and the catheter; and
a bulbous tip at a distal end of the internal elongated element, the bulbous tip having a maximum outer diameter that is at least as large as the inner diameter of the lumen of the catheter to enable a portion of the bulbous tip to seal against a distal end of the catheter.

14. The medical aspiration system of claim 13, wherein the internal elongated element includes at least one bend.

15. The medical aspiration system of claim 14, wherein the catheter includes at least one bend.

16. The medical aspiration system of claim 15, wherein the at least one bend of the internal elongated element is able to counteract the at least one bend of the catheter.

17. An aspiration method, comprising:
assembling an aspiration device with a guide wire in place within a subject's body;
introducing the aspiration device into the subject's body, along the guide wire;
applying an aspiration force to a catheter of the aspiration device;
moving an internal elongated element and a position of a bulbous tip of the aspiration device to selectively define an opening that communicates the aspiration force between the bulbous tip and a distal end of the catheter.

18. The method of claim 17, wherein moving the position of the bulbous tip enables aspiration through a side of an assembly including the catheter and the bulbous tip.

19. The method of claim 17, wherein moving the position of the bulbous tip enables aspiration around an entire circumference of a proximal side of the bulbous tip.

20. The method of claim 17, wherein moving the position of the bulbous tip includes back and forth movement of the bulbous tip.

21. The method of claim 20, wherein back and forth movement of the bulbous tip enables disruption of an aspirated substance.
